(19) United States
(12) Patent Application Publication
(10) Pub. No.: US 2022/0409759 A1
(43) Pub. Date: Dec. 29, 2022

(54) METHODS, SYSTEMS, APPARATUSES, AND DEVICES FOR FACILITATING SANITIZING INTERIOR SPACES OF AN AIRCRAFT USING A PRECONDITIONED AIR (PCA) SYSTEM

(71) Applicant: Michael Edward Cummings, Trinity, FL (US)

(72) Inventor: Michael Edward Cummings, Trinity, FL (US)

(21) Appl. No.: 17/849,781

(22) Filed: Jun. 27, 2022

Related U.S. Application Data
(60) Provisional application No. 63/259,208, filed on Jun. 27, 2021.

(51) Int. Cl. A61L 2/18; F16L 41/04; A61L 2/26; A61L 2/24; B64F 5/30

(52) U.S. Cl. CPC A61L 2/18; F16L 41/04; A61L 2/26; A61L 2/24; B64F 5/30; A61L 2202/11; A61L 2202/14; A61L 2202/25

(57) ABSTRACT

A method of facilitating sanitizing interior spaces of an aircraft using a pre-conditioned air (PCA) system. The method comprises cutting an opening in a duct wall of a pre-conditioned air (PCA) supply duct of the PCA system. Further, the method comprises installing a device in the PCA supply duct around the opening, coupling an inlet opening of the device with a device outlet port of a sanitizing device after the installing, wherein a sanitizing agent generator of the sanitizing device generates a sanitizing agent for transferring the sanitizing agent to the inlet opening through the device outlet port. Further, the method comprises injecting the sanitizing agent into the PCA supply duct from an outlet opening of the device based on the transferring. Further, the sanitizing agent mixes with the PCA based on the injecting. Further, the sanitizing agent is supplied to the interior space along with the PCA.

### CROSS-REFERENCE TO RELATED APPLICATION

This application claims the benefit of U.S. Provisional Patent Application No. 63/259,208, titled “Novel method for sanitizing aircraft interior space”, filed Jun. 27, 2021, which is incorporated by reference herein in its entirety.

### FIELD OF THE INVENTION

Generally, the present disclosure relates to the field of cleaning and sanitizing. More specifically, the present disclosure relates to methods, systems, apparatuses, and devices for facilitating sanitizing interior spaces of an aircraft using a pre-conditioned air (PCA) system.

### BACKGROUND OF THE INVENTION

Sanitizing aircraft is a rapidly developing area of interest that became the focus of government agencies and the air transport industry due to the global impacts of COVID-19. When global COVID-19 infection rates began to increase, the risk of human exposure to COVID-19 generated a very real fear and threat of infection for people gathering in enclosed spaces. While COVID-19 is used as an example for the preferred embodiment, this sanitization process is an effective method for sanitizing the interior of an aircraft against virtually any contagion threat within the enclosed space of an aircraft.

Due to the enclosed nature of air travel with passengers sitting inside the fuselage of an aircraft for extended periods of time, the airline industry rushed to deploy conventional methods of hand spraying, wiping down touch surfaces, and fogging aircraft cabin areas. These processes generally included cleaning personnel donning inconsistent levels of personal protective equipment (PPE) and applying various sanitizing agents to aircraft interiors and touch surfaces using conventional hand sprayers and foggers. To reduce manpower and exposure to potentially contaminated areas, in some cases robots and other specialized carts and equipment were sent into aircraft to sanitize cabin interiors including seats, tray tables, bathrooms, and other touch surfaces prior to crew and passengers entering the aircraft.

While airlines and stakeholders have improved the sanitizing and cleaning of aircraft interiors, the methods typically utilized are tedious, labor intensive and unnecessarily expose personnel to potential hazards from cleaning products and active biological and contagion threats due to incomplete sanitizing processes.

Therefore, there is a need for improved methods, systems, apparatuses, and devices for facilitating sanitizing interior spaces of an aircraft using a pre-conditioned air (PCA) system that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is a method of facilitating sanitizing interior spaces of an aircraft using a pre-conditioned air (PCA) system associated with the aircraft, in accordance with some embodiments. Accordingly, the method may include a step of cutting an opening in a duct wall of a pre-conditioned air (PCA) supply duct of the PCA system. Further, the PCA supply duct supplies a pre-conditioned air (PCA) to at least one interior space of the aircraft from a pre-conditioned air (PCA) generator of the PCA system. Further, the method may include a step of installing a device in the PCA supply duct around the opening. Further, the device may include a device body extending between a first end of the device and a second end of the device. Further, the device body may include an interior cavity extending between the first end and the second end. Further, the device may include an outlet opening disposed on a first end portion of the device body and an inlet opening disposed on a second end portion of the device body. Further, the interior cavity fluidly couples the inlet opening to the outlet opening. Further, the method may include a step of coupling the inlet opening of the device with a device outlet port of a sanitizing device after the installing. Further, a sanitizing agent generator of the sanitizing device may be configured for generating at least one sanitizing agent for transferring the at least one sanitizing agent to the inlet opening of the device through the device outlet port. Further, the method may include a step of injecting the at least one sanitizing agent into the PCA supply duct from the outlet opening of the device based on the transferring. Further, the at least one sanitizing agent mixes with the PCA based on the injecting. Further, the at least one sanitizing agent may be supplied to the at least one interior space along with the PCA for sanitizing the at least one interior space.

Further disclosed herein is a method of facilitating sanitizing interior spaces of an aircraft using a pre-conditioned air (PCA) system associated with the aircraft, in accordance with some embodiments. Accordingly, the method may include a step of cutting an opening in a duct wall of a pre-conditioned air (PCA) supply duct of the PCA system. Further, the PCA supply duct supplies a pre-conditioned air (PCA) to at least one interior space of the aircraft from a pre-conditioned air (PCA) generator of the PCA system. Further, the method may include a step of installing a device in the PCA supply duct around the opening. Further, the device may include a device body extending between a first end of the device and a second end of the device. Further, the device body may include an interior cavity extending between the first end and the second end. Further, the device may include an outlet opening disposed on a first end portion of the device body and an inlet opening disposed on a second end portion of the device body. Further, the interior cavity fluidly couples the inlet opening to the outlet opening. Further, the method may include a step of coupling the inlet opening of the device with a device outlet port of a sanitizing device after the installing. Further, a sanitizing agent generator of the sanitizing device may be configured for generating at least one sanitizing agent for transferring the at least one sanitizing agent to the inlet opening of the device through the device outlet port. Further, the method may include a step of injecting the at least one sanitizing agent into the PCA supply duct from the outlet opening of the device based on the transferring. Further, the at least one sanitizing agent mixes with the PCA based on the injecting. Further, the at least one sanitizing agent may be supplied to the at least one interior space along with the PCA for sanitizing the at least one interior space. Further, the method may include a step of receiving, using a communication unit of the sanitizing device, a contamination data associated with a contamination of the at least one interior space from at least one sensor disposed in the at least one interior space of the aircraft. Further, the method may include a step of analyzing, using a processing unit of the sanitizing device, the contamination data. Further, the method may include a step of determining, using the processing unit, at least one characteristic of the contamination of the at least one interior space based on the analyzing. Further, the method may include a step of determining, using the processing unit, at least one parameter for the at least one sanitizing agent based on the at least one characteristic. Further, the method may include a step of generating, using the processing unit, at least one command for the sanitizing agent generator based on the determining of the at least one parameter. Further, the at least one command corresponds to the at least one parameter. Further, the generating of the at least one sanitizing agent may be based on the at least one command.

Further, disclosed herein is a method of facilitating sanitizing interior spaces of an aircraft using a pre-conditioned air (PCA) system associated with the aircraft, in accordance with some embodiments. Accordingly, the method may include a step of cutting an opening in a duct wall of a pre-conditioned air (PCA) supply duct of the PCA system. Further, the PCA supply duct supplies a pre-conditioned air (PCA) to at least one interior space of the aircraft from a pre-conditioned air (PCA) generator of the PCA system. Further, the method may include a step of installing a device in the PCA supply duct around the opening. Further, the device may include a device body extending between a first end of the device and a second end of the device. Further, the device body may include an interior cavity extending between the first end and the second end. Further, the device may include an outlet opening disposed on a first end portion of the device body and an inlet opening disposed on a second end portion of the device body. Further, the interior cavity fluidly couples the inlet opening to the outlet opening. Further, the method may include a step of coupling the inlet opening of the device with a device outlet port of a sanitizing device after the installing. Further, a sanitizing agent generator of the sanitizing device may be configured for generating at least one sanitizing agent for transferring the at least one sanitizing agent to the inlet opening of the device through the device outlet port. Further, the method may include a step of injecting the at least one sanitizing agent into the PCA supply duct from the outlet opening of the device based on the transferring. Further, the at least one sanitizing agent mixes with the PCA based on the injecting. Further, the at least one sanitizing agent may be supplied to the at least one interior space along with the PCA for sanitizing the at least one interior space. Further, the method may include a step of receiving, using a communication unit of the PCA system, at least one first input. Further, the method may include a step of generating, using the pre-conditioned air (PCA) generator of the PCA system, the PCA based on the at least one first input. Further, the PCA supply duct supplies the PCA based on the generating of the PCA. Further, the PCA may include at least one amount of moisture. Further, the method may include a step of receiving, using the communication unit of the PCA system, at least one second input. Further, the method may include a step of generating, using the pre-conditioned air (PCA) generator, a first pre-conditioned air (PCA) based on the at least one second input. Further, the first PCA does not comprise the at least one amount of moisture. Further, the PCA supply duct supplies the first PCA to the at least one interior space of the aircraft based on the generating of the first PCA.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicant. In addition, the drawings may contain other marks owned by third parties and are being used for illustrative purposes only. All rights to various trademarks and copyrights represented herein, except those belonging to their respective owners, are vested in and the property of the applicant. The applicant retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is a flowchart of a method for facilitating sanitizing interior spaces of an aircraft using a pre-conditioned air (PCA) system associated with the aircraft, in accordance with some embodiments.

FIG. 2 is a flowchart of a method for installing the device into the PCA supply duct for facilitating the sanitizing of the interior spaces of the aircraft, in accordance with some embodiments.

FIG. 3 is a flowchart of a method for controlling the generating of the at least one sanitizing agent for facilitating the sanitizing of the interior spaces of the aircraft, in accordance with some embodiments.

FIG. 4 is a flowchart of a method for controlling the generating of the PCA for facilitating the sanitizing of the interior spaces of the aircraft, in accordance with some embodiments.

FIG. 5 is a flowchart of a method for facilitating sanitizing interior spaces of an aircraft using a pre-conditioned air (PCA) system associated with the aircraft, in accordance with some embodiments.

FIG. 6 is a continuation flowchart of the method for facilitating the sanitizing of the interior spaces of the aircraft, in accordance with some embodiments.

FIG. 7 is a flowchart of a method for facilitating sanitizing interior spaces of an aircraft using a pre-conditioned air (PCA) system associated with the aircraft, in accordance with some embodiments.

FIG. 8 is a continuation flowchart of the method for facilitating the sanitizing of the interior spaces of the aircraft, in accordance with some embodiments.

FIG. 9 is a schematic of a pre-conditioned air (PCA) system with an aircraft for facilitating sanitizing of interior spaces of the aircraft using the PCA system, in accordance with some embodiments.

FIG. 10 is a schematic of the pre-conditioned air (PCA) system and a sanitizing device with the aircraft for facilitating the sanitizing of the interior spaces of the aircraft using the PCA system, in accordance with some embodiments.

FIG. 11 is a front view of a device for facilitating sanitizing interior spaces of an aircraft using a pre-conditioned air (PCA) system associated with the aircraft, in accordance with some embodiments.

FIG. 12 is a top perspective view of the device, in accordance with some embodiments.

FIG. 13 is a bottom perspective view of the device, in accordance with some embodiments.

FIG. 14 is a right side cross-sectional view, along a section line C-D, of the device of FIG. 11, in accordance with some embodiments.

FIG. 15 is a left side cross-sectional view of the device, in accordance with some embodiments.

FIG. 16 is a front cross-sectional view, along a section line E-F, of the device of FIG. 15, in accordance with some embodiments.

FIG. 17 is an illustration of an online platform consistent with various embodiments of the present disclosure.

FIG. 18 is a block diagram of a computing device for implementing the methods disclosed herein, in accordance with some embodiments.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of sanitizing interior spaces of an aircraft using a pre-conditioned air (PCA) system, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor, and at least one actuator. Examples of the one or more client devices and/or the server computer may include, a desktop computer, a laptop computer, a tablet computer, a personal digital assistant, a portable electronic device, a wearable computer, a smartphone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server, etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface, etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the processing device may be a processing unit. Further, the server computer may include a communication device configured for communicating with one or more external devices. The one or more external devices may include, for example, but are not limited to, a client device, a third party database, a public database, a private database, and so on. Further, the communication device may be a communication unit, a communication interface, etc. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as but not limited to, data compression, data backup, data redundancy, deduplication, error correction, data finger-printing, role based access control, and so on.

Further, one or more steps of the method disclosed herein may be initiated, maintained, controlled, and/or terminated based on a control input received from one or more devices operated by one or more users such as, for example, but not limited to, an end user, an admin, a service provider, a service consumer, an agent, a broker and a representative thereof. Further, the user as defined herein may refer to a human unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer, etc.) and/or possession of a machine readable secret data (e.g. encryption key, decryption key, bar codes, etc.) and/or possession of one or more embodied characteristics unique to the user (e.g. biometric variables such as but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera, and so on. Likewise, the one or more steps may include receiving, using the communication device, the one or more embodied characteristics from one or more biometric sensors.

Further, one or more steps of the method may be automatically initiated, maintained, and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device, etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor, etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

### Overview

The present disclosure describes systems, methods, apparatuses, and devices for facilitating sanitizing interior spaces of an aircraft using a pre-conditioned air (PCA) system.

Further, the present disclosure describes a method for sanitizing the interior of an aircraft fuselage from outside the aircraft. More particularly, the method is a process of introducing an airborne sanitizing agent into the aircraft's existing pre-conditioned air (PCA) intake system. The method incorporates a custom sanitizing agent injection device hereafter referred to as the “device” installed in the PCA supply ductwork that introduces sanitizing agent into the flow of PCA from the exterior of the aircraft. The PCA mixed with sanitizing agent sanitizes the HVAC ductwork and blankets the interior of the aircraft destroying contaminants throughout the cabin.

This process allows the aircraft interior to be sanitized without having to enter the fuselage prior to or during the sanitizing process. While the preferred embodiment custom device is described as being installed in the PCA intake ductwork it could also be adapted to a coupling that connects to an aircraft's PCA intake port or ports.

Further, the present disclosure describes a process for sanitizing aircraft interior cabin areas by combining airborne sanitizing agents into the flow of the aircraft's existing pre-conditioned air (PCA) intake system prior to dispersing PCA through the aircraft's internal HVAC ductwork and interior cabin ventilation system.

Further, the process allows the aircraft's cabin areas to be sanitized without entering the fuselage thereby eliminating the risk of exposure to contamination that may be present inside the aircraft. The process also reduces the amount of wasted chemicals and overly wet seats and touch surfaces often caused by inconsistent amounts of human sprayed and/or fogged application of sanitizing products. The extensive personal protective equipment (PPE) required to enter a potentially contaminated aircraft is reduced due to the need to use only PPE required to safely work outside the aircraft with sanitizing agent and equipment being used for this process.

Parked aircraft are typically connected to an external PCA supply using a flexible duct that extends from the PCA source to the aircraft's low pressure PCA inlet port typically located on the exterior underside of the fuselage. When the PCA supply duct coupling is connected to the aircraft's low pressure intake, externally supplied PCA flows from the external PCA source through flexible ductwork and into the aircraft's low pressure PCA port for distribution via the aircraft's existing ductwork and finally into the cabin space.

Further, the present disclosure describes a device designed to introduce a sanitizing agent into the external PCA supply ductwork. Sanitizing agent device with flow shield. The curved PCA flow shield allows the sanitizing agent to exit the device orifice and mixes with the PCA moving through the aircraft's PCA supply duct. Further, the interior flange includes a directional arrow to assist an installer with proper installation orientation. The body of the supply duct is connected to the device and the supply duct allows supplying the aircraft with PCA.

Further, the sanitizing agent is introduced into the intake side of the device through a quick release coupling connected to the equipment producing the sanitizing agent to be introduced through the device and into the PCA flow via the orifice. The sanitizing agent enters from the sanitizing generating equipment through the bottom of the quick release coupling and flows upward through the device and out into the PCA flow via an orifice. PCA flow direction is perpendicular to the device. The male threaded body of the device allows for the female threaded custom nut with thumb tabs to hand tighten the threaded nut onto the PCA supply duct. The threaded body of the device allows for a female threaded custom nut with thumb tabs to hand tighten the nozzle onto the PCA supply duct. The male threaded body of the device allows for the female threaded custom nut with thumb tabs to hand tighten the threaded nut onto the PCA supply duct.

Further, the present disclosure describes a sanitizing process, the following are best practice recommendations to be followed prior to sanitizing the aircraft cabin. Items 1, 2, and 4 are required irrespective of whether this sanitizing process is utilized.

1. Remove trash and any spilled items and/or bodily fluids from surfaces inside the aircraft cabin.

2. Lavatories must be pre-cleaned and galley trash and food spills removed.

3. For deep sanitizing, galley and all interior doors and cabin doors in an open position during aircraft sanitizing treatment. This requirement will vary based on the type of equipment utilized for sanitizing the aircraft interior as some equipment (i.e. cold sterilant foggers) has the ability to convert sanitizing agent to particle sizes small enough to pass through door jambs and cabinet door openings and enter overhead bins without having to leave doors open.

4. Special attention to the cleanup of any biological waste or remains (i.e. blood, feces, body tissue, vomit, needles, surgical items, etc.) shall be removed, discarded, and treated per requirements of the local authority having jurisdiction prior to sanitizing aircraft.

5. Prior to exiting the aircraft and starting the sanitizing process it is recommended but not required that all air vents are open above passenger seating. The more vents that are left open the more effectively sanitizing agent will disperse throughout the aircraft cabin. This may be requested by the flight crew during landing for passenger assistance in opening the ventilation system in preparation for sanitizing the cabin interior.

6. Seatback trays and overhead bins ideally remain in the open position during aircraft sanitization.

This method of sanitizing an aircraft cabin introduces sanitizing agent into the external PCA supply ductwork via a custom device installed between the outflow of the PCA generating equipment and prior to airflow entering the aircraft's existing low pressure PCA inlet port. The device is installed through a circular opening cut into the wall of the existing PCA supply duct connected to the aircraft's existing low-pressure PCA inlet port. The preferred embodiment device is ideally installed as close to the aircraft's low pressure PCA inlet port as possible to reduce the distance sanitizing agent must travel through the exterior PCA ductwork prior to reaching surfaces inside the cabin interior. Minimizing the PCA flow distance maximizes the efficacy of the sanitizing agent as it flows through the aircraft's ductwork system and provides a fine coating of the sanitizing agent throughout the aircraft ductwork and cabin interior.

This process results in the aircraft's ductwork and cabin areas being sanitized from outside the aircraft and other than the addition of the preferred embodiment device, no special upgrades are required to ramp or passenger loading bridge based PCA equipment. While the preferred embodiment utilizes the preferred embodiment device for installation in the aircraft's PCA flexible supply duct, a similarly designed alternate embodiment of the device can be installed at any point along the low pressure PCA intake servicing the aircraft although it is recommended the nozzle is installed as close as possible to the low pressure PCA intake port on the aircraft as possible to minimize the time required for sanitizing agent to enter the aircraft HVAC ductwork and maximize the effectiveness of the sanitizing agent.

The sanitizing agent is introduced into the PCA flexible duct through the installed device. As PCA flows through the flexible duct toward the aircraft's existing low pressure PCA port, sanitizing agent exiting the orifice inside the PCA supply duct is introduced and combined with the PCA flow where the interior space of the PCA supply flexible duct acts as a mixing chamber. The combined PCA mixed with the sanitizing agent flows through the flexible duct, then passes through the aircraft's existing PCA low pressure port, and PCA combined with the sanitizing agent is then dispersed through the aircraft's existing HVAC ductwork and into the cabin. As PCA combined with sanitizing agent flows through the aircraft's existing ductwork, PCA exits at each duct diffuser and blankets the aircraft interior with sanitizing product.

Aircraft interiors and HVAC systems are designed to withstand exposure to the interior fog that naturally and frequently occurs when warm humid air inside an aircraft cabin mixes with cool air from the aircraft's air conditioning system. As the cool air exiting the interior cabin vents mixes with hot humid air inside the cabin the humidity condenses and results in a fog of water vapor that blankets the cabin interior. This is a very common occurrence when aircraft depart in warmer climates where hot humid air from outside the fuselage enters the cabin through an open door causing a fog to form and blanket the cabin interior. In the case of this patent sanitizing agent is introduced with the aircraft's onboard air conditioning turned off while preconditioned air combined with sanitizing agent flows through the aircraft as described above.

The present disclosure describes using of sanitizing agents which are not limited to a particular sanitizing agent or chemical and function with sanitizing agents introduced in a variety of particle sizes generated by readily available fogging and spray equipment. The present disclosure describes allowing airport stakeholders to continue using their existing PCA equipment while taking advantage of the latest sanitizing agents not only to prevent the spread of disease resulting from present and future biological threats.

Further, the present disclosure describes a method for sanitizing agent introduction and cabin exposure verification. This method for sanitizing an aircraft cabin uses the preconditioned air mixed with a sanitizing agent to coat the interior of the aircraft ductwork and throughout the interior of the aircraft cabin thereby blanketing the space in sanitizing agent for a thorough and efficient application of the sanitizing agent. Sanitizing agent application rates, particle sizes, and durations may be adjusted as necessary to achieve various levels of treatment.

To document the efficacy of this process, sanitizing agent exposure to surfaces throughout the aircraft cabin may be verified using various testing procedures but the preferred embodiment for this patent utilizes readily available indicator test strips that activate when exposed to the sanitizing agent utilized. Test indicator strips are left on surfaces including tray tables, seats, and common touch surfaces to provide a verifiable method of confirming process effectiveness.

The present disclosure describes an aircraft cabin sanitizing process which includes disabling the preconditioned air compressor(s) and any dehumidification during the sanitizing process allowing the sanitizing agent to remain in a micro droplet/aerosol state throughout the process. This is accomplished by setting the PCA unit to only blow fresh air into the aircraft vs conditioned air. As soon as sanitizing is complete based on the requirements of the specific sanitizing agent utilized the PCA unit is switched into conditioned air mode, and dehumidification of the cabin takes place thereby evaporating any residual sanitizing agent in the aircraft cabin. The preferred embodiment for this patent utilizes a hydrogen peroxide solution mixed with peracetic acid which decays rapidly into the water with any residual water evaporating very rapidly once the PCA system is set into conditioned air mode and incoming PCA begins dehumidifying the space. PCA compressor(s) may operate or be deactivated during introduction of sanitizing agent into the aircraft based on the requirements of the particular sanitizing agent being utilized.

Throughout the sanitizing process, the external PCA unit operates as normal with one of the aircraft doors open to provide a means of expelling air from inside the cabin as fresh air enters the space mixed with a sanitizing agent via the PCA distribution system.

Further, the present disclosure describes a sanitizing agent injection port device (the “device”). Further, each of the elements of the device is configured with at least one interior flange with an orifice serving as an injection port that is centered on the interior flange fabricated with a threaded body on the outside portion of the injection port flange, configured to accept a threaded nut with a round head with tabs protruding from the outer face of the nut. The interior face of the injection port flange in this preferred embodiment has a rounded projection that extends out into the PCA flow thereby allowing the sanitizing agent to exit the opening in the injection port flange and disperse into the PCA as it flows toward the aircraft's low pressure PCA inlet.

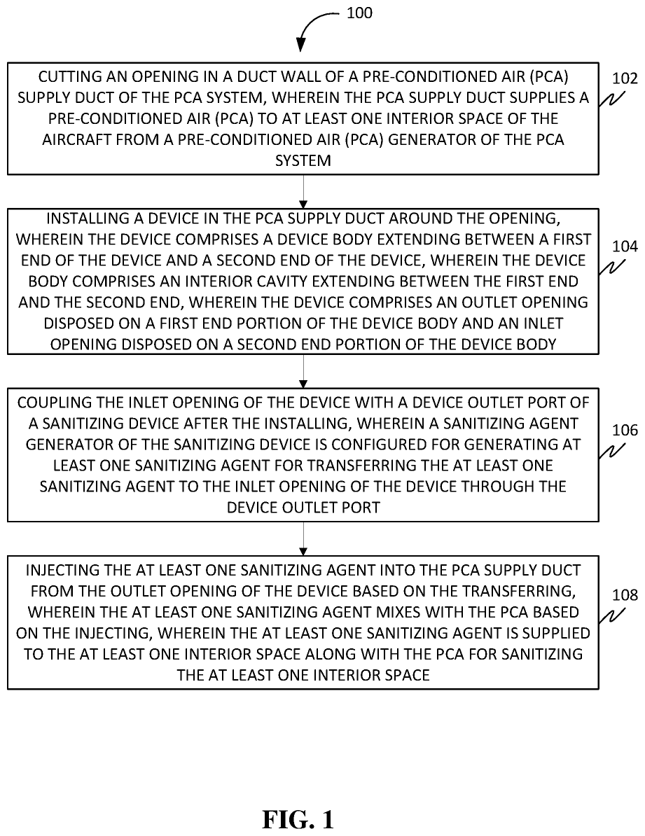

FIG. 1 is a flowchart of a method 100 for facilitating sanitizing interior spaces of an aircraft using a pre-conditioned air (PCA) system associated with the aircraft, in accordance with some embodiments. Accordingly, at 102, the method 100 may include cutting an opening in a duct wall of a pre-conditioned air (PCA) supply duct of the PCA system. Further, the PCA supply duct supplies a pre-conditioned air (PCA) to at least one interior space of the aircraft from a pre-conditioned air (PCA) generator of the PCA system. Further, the aircraft may be on the ground.

Further, at 104, the method 100 may include installing a device in the PCA supply duct around the opening. Further, the device may include a device body extending between a first end of the device and a second end of the device. Further, the device body may include an interior cavity extending between the first end and the second end. Further, the device may include an outlet opening disposed on a first end portion of the device body and an inlet opening disposed on a second end portion of the device body. Further, the interior cavity fluidly couples the inlet opening to the outlet opening. Further, the first end portion may be placed inside the PCA supply duct and the second end portion may be placed outside the PCA supply duct based on the installing.

Further, at 106, the method 100 may include coupling the inlet opening of the device with a device outlet port of a sanitizing device after the installing. Further, a sanitizing agent generator of the sanitizing device may be configured for generating at least one sanitizing agent for transferring the at least one sanitizing agent to the inlet opening of the device through the device outlet port.

Further, at 108, the method 100 may include injecting the at least one sanitizing agent into the PCA supply duct from the outlet opening of the device based on the transferring. Further, the at least one sanitizing agent mixes with the PCA based on the injecting. Further, the at least one sanitizing agent may be supplied to the at least one interior space along with the PCA for sanitizing the at least one interior space.

Further, in some embodiments, the device may include a cutting tool disposed on the first end portion. Further, the cutting tool may be used for the cutting of the opening in the duct wall.

Further, in some embodiments, the coupling of the inlet opening with the device outlet port of the sanitizing device may include engaging the device outlet port of the sanitizing device to a quick release coupling of the device. Further, the quick release coupling may be disposed on the second end portion.

In further embodiments, the method 100 may include removing at least one waste from the at least one interior space of the aircraft. Further, the injecting of the at least one sanitizing agent into the PCA supply duct may be based on the removing of the at least one waste.

In further embodiments, the method 100 may include opening a door of each of at least one cabinet disposed in the at least one interior space of the aircraft. Further, the injecting of the at least one sanitizing agent into the PCA supply duct may be based on the opening of the door of each of the at least one cabinet.

In further embodiments, the method 100 may include transitioning at least one ventilation system (such as a ventilation system 910) of the aircraft to an open state. Further, the injecting of the at least one sanitizing agent into the PCA supply duct may be based on the transitioning of the ventilation system to the open state.

In further embodiments, the method 100 may include switching an air conditioning system (such as an air conditioning system 912) of the aircraft to an off state. Further, the injecting of the at least one sanitizing agent into the PCA supply duct may be based on the switching of the air conditioning system to the off state. Further, the switching of the air conditioning system to the off state prevents dehumidification of the at least one interior space.

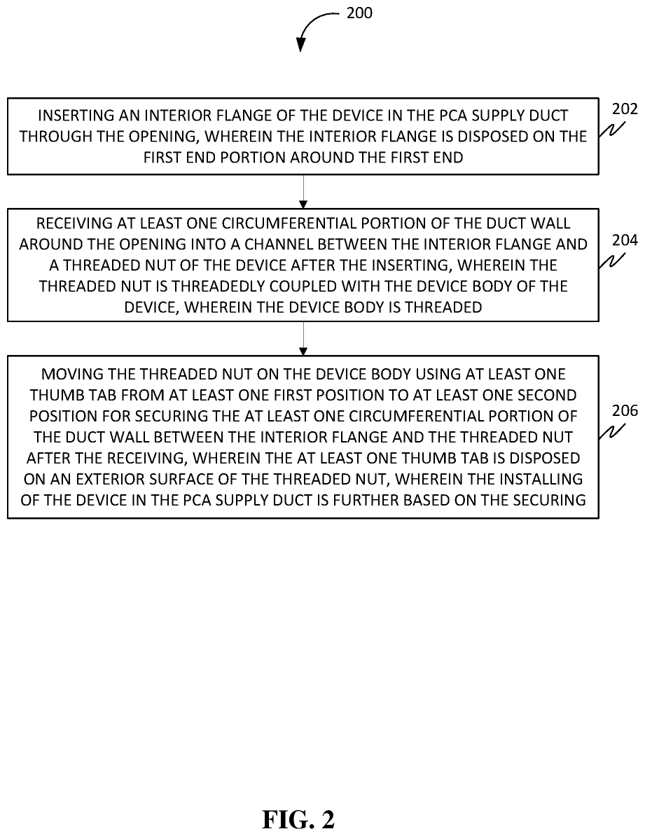

FIG. 2 is a flowchart of a method 200 for installing the device into the PCA supply duct for facilitating the sanitizing of the interior spaces of the aircraft, in accordance with some embodiments. Accordingly, at 202, the method 200 may include inserting an interior flange of the device in the PCA supply duct through the opening. Further, the interior flange may be disposed on the first end portion around the first end.

Further, at 204, the method 200 may include receiving at least one circumferential portion of the duct wall around the opening into a channel between the interior flange and a threaded nut of the device after the inserting. Further, the threaded nut may be threadedly coupled with the device body of the device. Further, the device body may be threaded.

Further, at 206, the method 200 may include moving the threaded nut on the device body using at least one thumb tab from at least one first position to at least one second position for securing the at least one circumferential portion of the duct wall between the interior flange and the threaded nut after the receiving. Further, the at least one thumb tab may be disposed on an exterior surface of the threaded nut. Further, the installing of the device in the PCA supply duct may be based on the securing.

In further embodiments, the method 200 may include rotating the device between a plurality of positions after the inserting of the interior flange for orienting a flow shield of the device towards an inlet port of the aircraft. Further, the flow shield directs a flow of the at least one sanitizing agent in a direction. Further, the orienting of the flow shield orients the flow of the at least one sanitizing agent parallel to a flow of the PCA. Further, the flow shield may be disposed on the first end portion around at least a part of a periphery of the outlet opening. Further, a duct outlet of the PCA supply duct may be coupled to the inlet port of the aircraft. Further, the moving of the threaded nut on the device body may be based on the rotating.

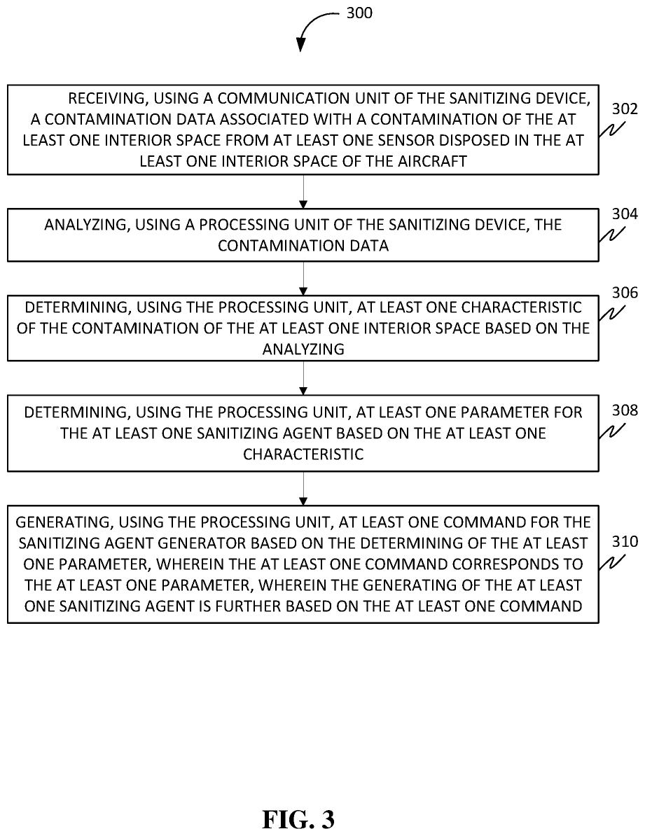

FIG. 3 is a flowchart of a method 300 for controlling the generating of the at least one sanitizing agent for facilitating the sanitizing of the interior spaces of the aircraft, in accordance with some embodiments. Accordingly, at 302, the method 300 may include receiving, using a communication unit of the sanitizing device, a contamination data associated with a contamination of the at least one interior space from at least one sensor disposed in the at least one interior space of the aircraft.

Further, at 304, the method 300 may include analyzing, using a processing unit of the sanitizing device, the contamination data.

Further, at 306, the method 300 may include determining, using the processing unit of the sanitizing device, at least one characteristic of the contamination of the at least one interior space based on the analyzing.

Further, at 308, the method 300 may include determining, using the processing unit of the sanitizing device, at least one parameter for the at least one sanitizing agent based on the at least one characteristic.

Further, at 310, the method 300 may include generating, using the processing unit of the sanitizing device, at least one command for the sanitizing agent generator based on the determining of the at least one parameter. Further, the at least one command corresponds to the at least one parameter. Further, the generating of the at least one sanitizing agent may be based on the at least one command.

Further, in some embodiments, the at least one parameter may include a droplet size of a plurality of droplets of the at least one sanitizing agent. Further, the generating of the at least one command may be based on the droplet size of the plurality of droplets of the at least one sanitizing agent. Further, the generating of the at least one sanitizing agent may include generating the plurality of droplets of the at least one sanitizing agent having the droplet size based on the at least one command.

Further, in some embodiments, Further, the at least one parameter may include an application duration of the at least one sanitizing agent. Further, the generating of the at least one command may be based on the application duration of the at least one sanitizing agent. Further, the generating of the at least one sanitizing agent may include generating the at least one sanitizing agent for the application duration based on the at least one command.

Further, in some embodiments, Further, the at least one parameter may include a sanitizing agent identifier of a sanitizing agent of the at least one sanitizing agent. Further, the generating of the at least one command may be based on the sanitizing agent identifier. Further, the generating of the at least one sanitizing agent may include generating the sanitizing agent based on the at least one command.

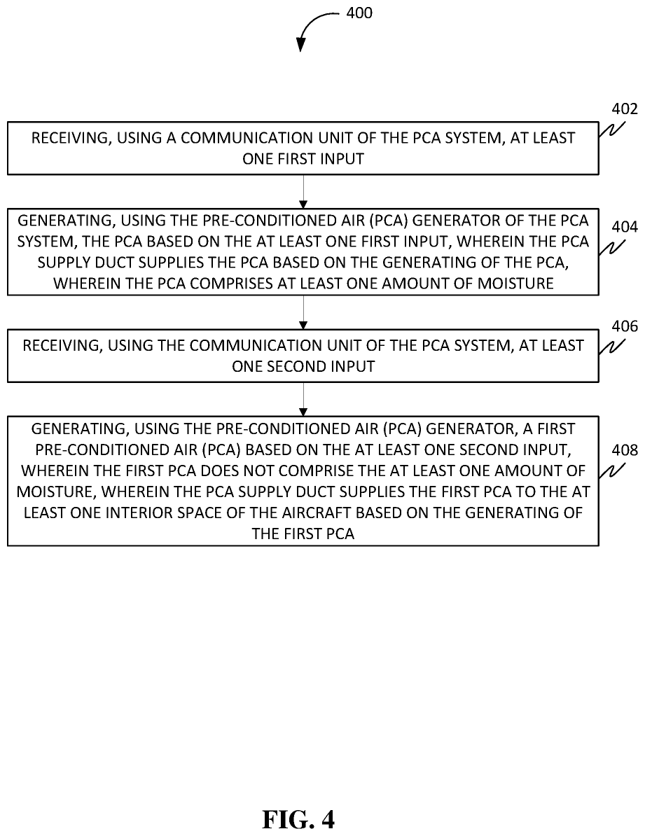

FIG. 4 is a flowchart of a method 400 for controlling the generating of the PCA for facilitating the sanitizing of the interior spaces of the aircraft, in accordance with some embodiments. Accordingly, at 402, the method 400 may include receiving, using a communication unit of the PCA system, at least one first input.

Further, at 404, the method 400 may include generating, using the pre-conditioned air (PCA) generator of the PCA system, the PCA based on the at least one first input. Further, the PCA supply duct supplies the PCA based on the generating of the PCA. Further, the PCA may include at least one amount of moisture.

Further, at 406, the method 400 may include receiving, using the communication unit of the PCA system, at least one second input.

Further, at 408, the method 400 may include generating, using the pre-conditioned air (PCA) generator, a first pre-conditioned air (PCA) based on the at least one second input. Further, the first PCA does not comprise the at least one amount of moisture. Further, the PCA supply duct supplies the first PCA to the at least one interior space of the aircraft based on the generating of the first PCA.

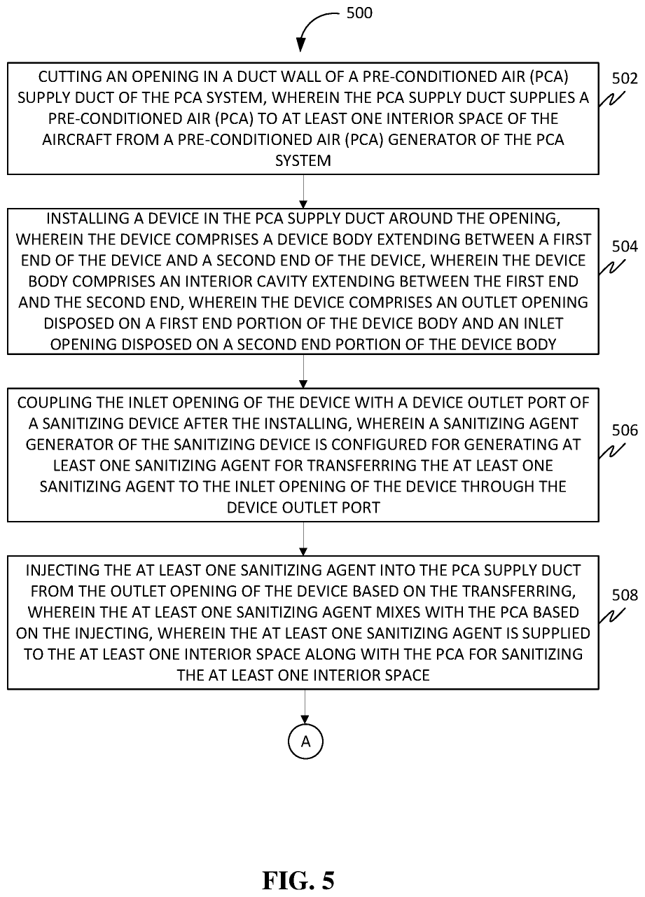

FIG. 5 is a flowchart of a method 500 for facilitating sanitizing interior spaces of an aircraft using a pre-conditioned air (PCA) system associated with the aircraft, in accordance with some embodiments. Accordingly, at 502, the method 500 may include cutting an opening in a duct wall of a pre-conditioned air (PCA) supply duct of the PCA system. Further, the PCA supply duct supplies a pre-conditioned air (PCA) to at least one interior space of the aircraft from a pre-conditioned air (PCA) generator of the PCA system. Further, the aircraft may be on the ground.

Further, at 504, the method 500 may include installing a device in the PCA supply duct around the opening. Further, the device may include a device body extending between a first end of the device and a second end of the device. Further, the device body may include an interior cavity extending between the first end and the second end. Further, the device may include an outlet opening disposed on a first end portion of the device body and an inlet opening disposed on a second end portion of the device body. Further, the interior cavity fluidly couples the inlet opening to the outlet opening. Further, the first end portion may be placed inside the PCA supply duct and the second end portion may be placed outside the PCA supply duct based on the installing.

Further, at 506, the method 500 may include coupling the inlet opening of the device with a device outlet port of a sanitizing device after the installing. Further, a sanitizing agent generator of the sanitizing device may be configured for generating at least one sanitizing agent for transferring the at least one sanitizing agent to the inlet opening of the device through the device outlet port.

Further, at 508, the method 500 may include injecting the at least one sanitizing agent into the PCA supply duct from the outlet opening of the device based on the transferring. Further, the at least one sanitizing agent mixes with the PCA based on the injecting. Further, the at least one sanitizing agent may be supplied to the at least one interior space along with the PCA for sanitizing the at least one interior space.

Further, at 510, the method 500 may include receiving, using a communication unit of the sanitizing device, a contamination data associated with a contamination of the at least one interior space from at least one sensor disposed in the at least one interior space of the aircraft.

Further, at 512, the method 500 may include analyzing, using a processing unit of the sanitizing device, the contamination data.

Further, at 514, the method 500 may include determining, using the processing unit of the sanitizing device, at least one characteristic of the contamination of the at least one interior space based on the analyzing.

Further, at 516, the method 500 may include determining, using the processing unit of the sanitizing device, at least one parameter for the at least one sanitizing agent based on the at least one characteristic.

Further, at 518, the method 500 may include generating, using the processing unit of the sanitizing device, at least one command for the sanitizing agent generator based on the determining of the at least one parameter. Further, the at least one command corresponds to the at least one parameter. Further, the generating of the at least one sanitizing agent may be based on the at least one command.

Further, in some embodiments, the coupling of the inlet opening with the device outlet port of the sanitizing device may include engaging the device outlet port of the sanitizing device to a quick release coupling of the device. Further, the quick release coupling may be disposed on the second end portion.

In further embodiments, the method 500 may include opening a door of each of at least one cabinet disposed in the at least one interior space of the aircraft. Further, the injecting of the at least one sanitizing agent into the PCA supply duct may be based on the opening of the door of each of the at least one cabinet.

In further embodiments, the method 500 may include transitioning at least one ventilation system of the aircraft to an open state. Further, the injecting of the at least one sanitizing agent into the PCA supply duct may be based on the transitioning of the ventilation system to the open state.

In further embodiments, the method 500 may include switching an air conditioning system of the aircraft to an off state. Further, the injecting of the at least one sanitizing agent into the PCA supply duct may be based on the switching of the air conditioning system to the off state.

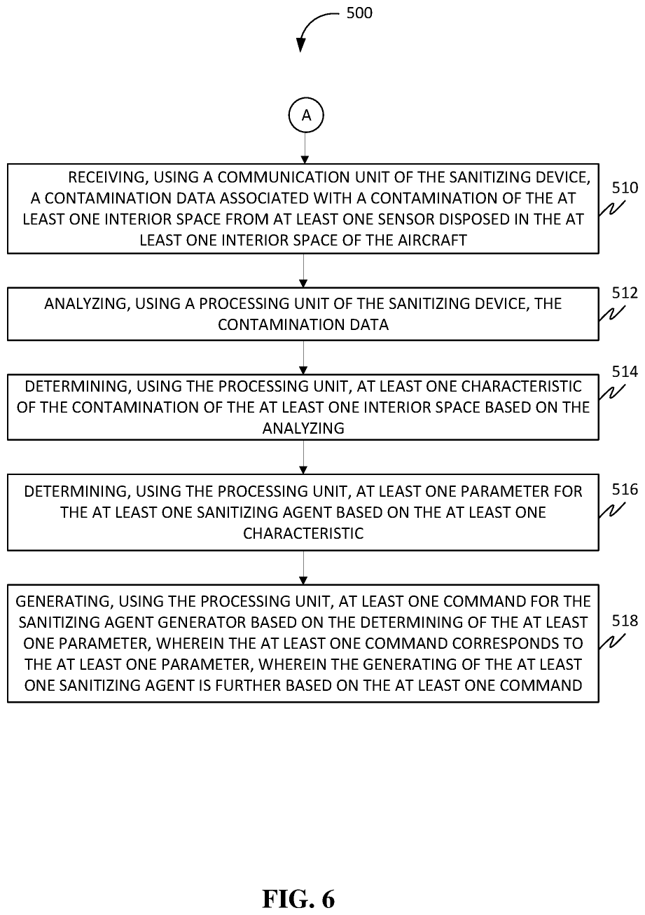

FIG. 6 is a continuation flowchart of the method 500 for facilitating the sanitizing of the interior spaces of the aircraft, in accordance with some embodiments.

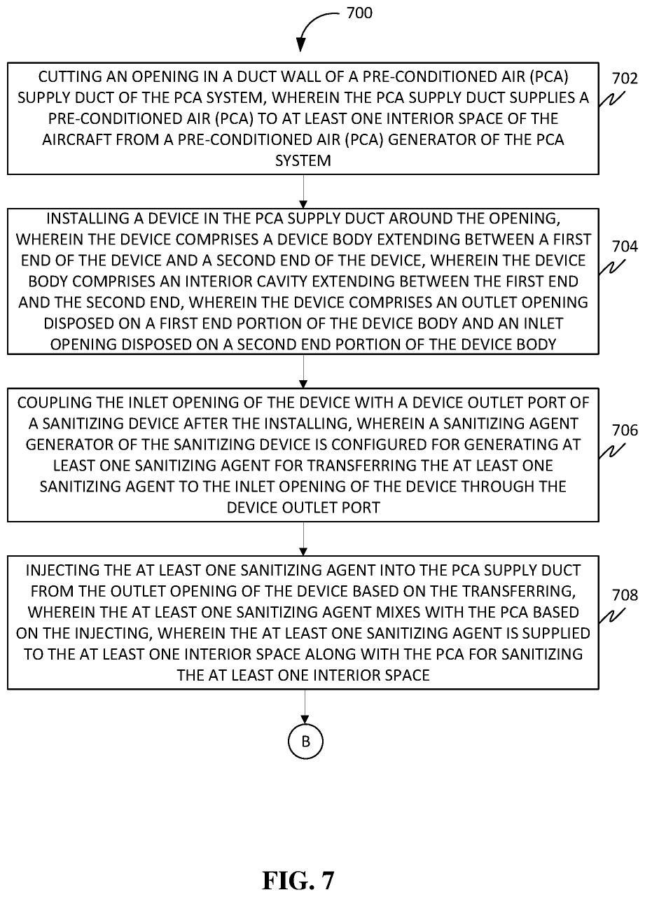

FIG. 7 is a flowchart of a method 700 for facilitating sanitizing interior spaces of an aircraft using a pre-conditioned air (PCA) system associated with the aircraft, in accordance with some embodiments. Accordingly, at 702, the method 700 may include cutting an opening in a duct wall of a pre-conditioned air (PCA) supply duct of the PCA system. Further, the PCA supply duct supplies a pre-conditioned air (PCA) to at least one interior space of the aircraft from a pre-conditioned air (PCA) generator of the PCA system. Further, the aircraft may be on the ground.

Further, at 704, the method 700 may include installing a device in the PCA supply duct around the opening. Further, the device may include a device body extending between a first end of the device and a second end of the device. Further, the device body may include an interior cavity extending between the first end and the second end. Further, the device may include an outlet opening disposed on a first end portion of the device body and an inlet opening disposed on a second end portion of the device body. Further, the interior cavity fluidly couples the inlet opening to the outlet opening. Further, the first end portion may be placed inside the PCA supply duct and the second end portion may be placed outside the PCA supply duct based on the installing.

Further, at 706, the method 700 may include coupling the inlet opening of the device with a device outlet port of a sanitizing device after the installing. Further, a sanitizing agent generator of the sanitizing device may be configured for generating at least one sanitizing agent for transferring the at least one sanitizing agent to the inlet opening of the device through the device outlet port.

Further, at 708, the method 700 may include injecting the at least one sanitizing agent into the PCA supply duct from the outlet opening of the device based on the transferring. Further, the at least one sanitizing agent mixes with the PCA based on the injecting. Further, the at least one sanitizing agent may be supplied to the at least one interior space along with the PCA for sanitizing the at least one interior space.

Further, at 710, the method 700 may include receiving, using a communication unit of the PCA system, at least one first input.

Further, at 712, the method 700 may include generating, using the pre-conditioned air (PCA) generator of the PCA system, the PCA based on the at least one first input. Further, the PCA supply duct supplies the PCA based on the generating of the PCA. Further, the PCA may include at least one amount of moisture.

Further, at 714, the method 700 may include receiving, using the communication unit of the PCA system, at least one second input.

Further, at 716, the method 700 may include generating, using the pre-conditioned air (PCA) generator, a first pre-conditioned air (PCA) based on the at least one second input.

Further, the first PCA does not comprise the at least one amount of moisture. Further, the PCA supply duct supplies the first PCA to the at least one interior space of the aircraft based on the generating of the first PCA.

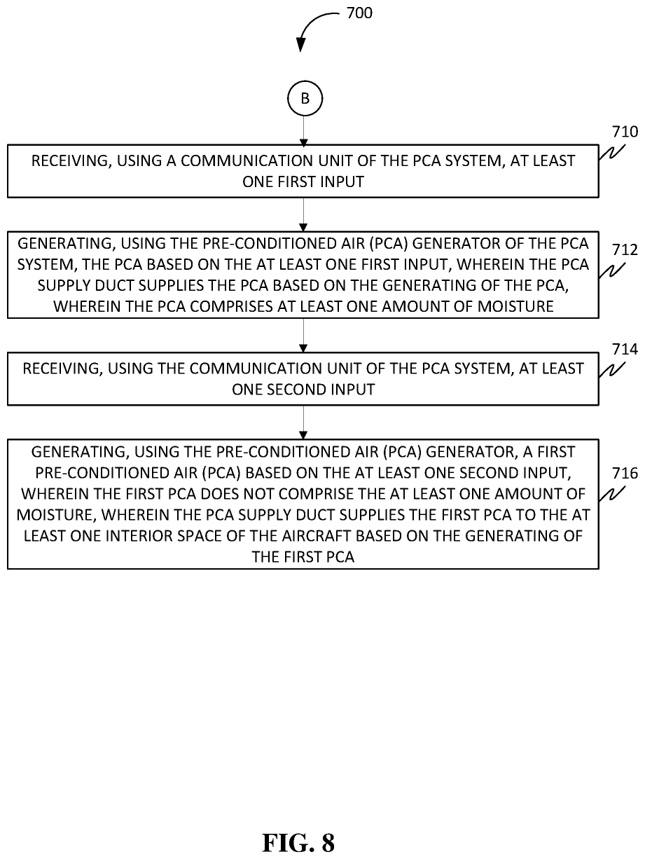

FIG. 8 is a continuation flowchart of the method 700 for facilitating the sanitizing of the interior spaces of the aircraft, in accordance with some embodiments.

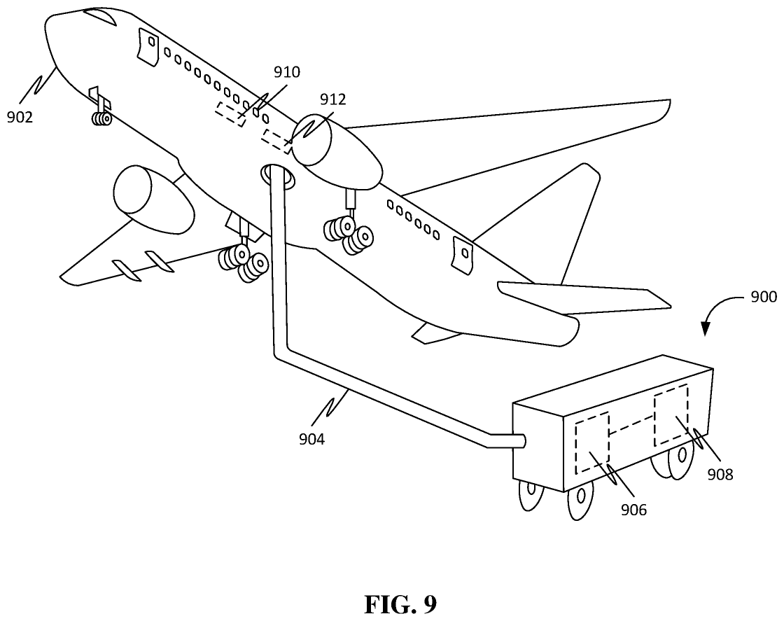

FIG. 9 is a schematic of a pre-conditioned air (PCA) system 900 with an aircraft 902 for facilitating sanitizing of interior spaces of the aircraft 902 using the PCA system 900, in accordance with some embodiments. Further, the aircraft 902 may include a ventilation system 910 and an air conditioning system 912. Further, the PCA system 900 may include a pre-conditioned air (PCA) supply duct 904. Further, the PCA supply duct 904 supplies a pre-conditioned air (PCA) to at least one interior space of the aircraft 902.

Further, in some embodiments, the PCA system 900 may include a communication unit 906. Further, the communication unit 906 of the PCA system 900 may be configured for receiving at least one first input. Further, the at least one first input may be received from at least one client device, an input device, etc. Further, the communication unit 906 of the PCA system 900 may be configured for receiving at least one second input. Further, the at least one second input may be received from the at least one client device, the input device, etc. Further, the PCA system 900 may include a pre-conditioned air (PCA) generator 908. Further, the pre-conditioned air (PCA) generator 908 may be communicatively coupled with the communication unit 906 of the PCA system 900. Further, the pre-conditioned air (PCA) generator 908 of the PCA system 900 may be configured for generating the PCA based on the at least one first input. Further, the PCA supply duct 904 supplies the PCA based on the generating of the PCA. Further, the PCA may include at least one amount of moisture. Further, the pre-conditioned air (PCA) generator 908 of the PCA system 900 may be configured for generating a first pre-conditioned air (PCA) based on the at least one second input. Further, the first PCA does not comprise the at least one amount of moisture. Further, the PCA supply duct 904 supplies the first PCA to the at least one interior space of the aircraft 902 based on the generating of the first PCA.

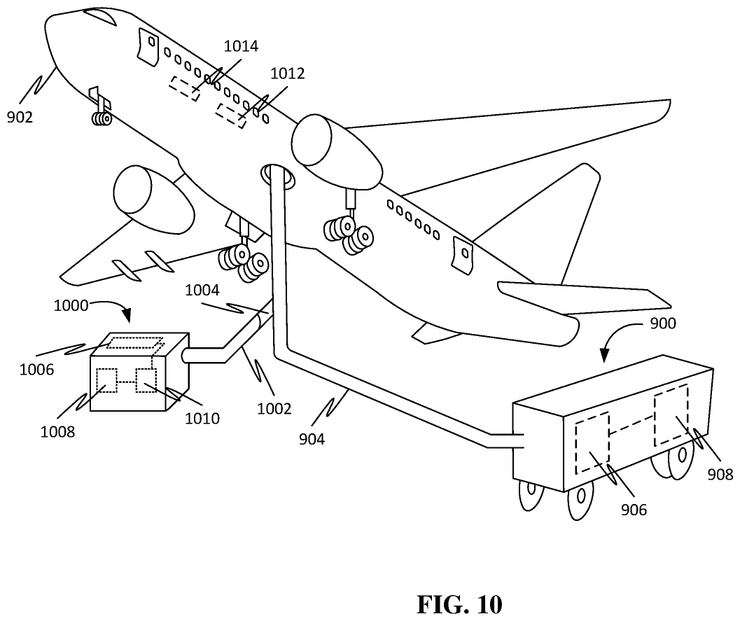

FIG. 10 is a schematic of the pre-conditioned air (PCA) system 900 and a sanitizing device 1000 with the aircraft 902 for facilitating the sanitizing of the interior spaces of the aircraft 902 using the PCA system 900, in accordance with some embodiments. Further, a device outlet port 1002 of the sanitizing device 1000 may be coupled with an inlet opening of a device 1004. Further, a sanitizing agent generator 1006 of the sanitizing device 1000 may be configured for generating at least one sanitizing agent for transferring the at least one sanitizing agent to the inlet opening of the device 1004 through the device outlet port 1002. Further, the at least one sanitizing agent may be injected into the PCA supply duct 904 from an outlet opening of the device 1004 based on the transferring. Further, the at least one sanitizing agent mixes with the PCA based on the injecting. Further, the at least one sanitizing agent may be supplied to the at least one interior space along with the PCA for sanitizing the at least one interior space.

Further, in some embodiments, the sanitizing device 1000 may include a communication unit 1008. Further, the communication unit 1008 of the sanitizing device 1000 may be configured for receiving a contamination data associated with a contamination of the at least one interior space from at least one sensor 1012 disposed in the at least one interior space of the aircraft 902. Further, the at least one sensor 1012 may be configured for generating the contamination data based on detecting at least one contaminant present in the at least one interior space. Further, the at least one contaminant may include a chemical contaminant, a biological contaminant, a solid contaminant, a liquid contaminant, a gaseous contaminant, etc. Further, the at least one sensor 1012 may include a camera, a chemical sensor, a biological sensor, an air quality sensor, an environmental sensor, etc. Further, the sanitizing device 1000 may include a processing unit 1010 communicatively coupled with the communication unit 1008 of the sanitizing device 1000. Further, the processing unit 1010 of the sanitizing device 1000 may be configured for analyzing the contamination data. Further, the processing unit 1010 of the sanitizing device 1000 may be configured for determining at least one characteristic of the contamination of the at least one interior space based on the analyzing. Further, the at least one characteristic of the contamination may include a type of the contamination, a toxicity of the contamination, a nature of the contamination, etc. Further, the processing unit 1010 of the sanitizing device 1000 may be configured for determining at least one parameter for the at least one sanitizing agent based on the at least one characteristic. Further, the at least one parameter may include a duration of application of the at least one sanitizing agent, a type of the at least one sanitizing agent, an amount of the at least one sanitizing agent, etc. Further, the processing unit 1010 of the sanitizing device 1000 may be configured for generating at least one command for the sanitizing agent generator 1006 based on the determining of the at least one parameter. Further, the at least one command corresponds to the at least one parameter. Further, the processing unit 1010 of the sanitizing device 1000 may be communicatively coupled with the sanitizing agent generator 1006. Further, the generating of the at least one sanitizing agent may be further based on the at least one command.

Further, in an embodiment, the processing unit 1010 of the sanitizing device 1000 may be configured for generating the at least one first input based on the at least one parameter. Further, the communication unit 1008 of the sanitizing device 1000 may be configured for transmitting the at least one first input to the communication unit 906 of the PCA system 900.

Further, in an embodiment, the communication unit 1008 of the sanitizing device 1000 may be configured for receiving a sanitizing data associated with the sanitizing of the at least one interior space from at least one first sensor 1014 disposed in the at least one interior space of the aircraft 902. Further, the at least one first sensor 1014 may be configured for generating the sanitation data based on detecting an amount of the at least one sanitizing agent present in the at least one interior space. Further, the at least one first sensor 1014 may include a chemical sensor, an environment sensor, etc. Further, the processing unit 1010 of the sanitizing device 1000 may be configured for analyzing the sanitation data. Further, the processing unit 1010 of the sanitizing device 1000 may be configured for determining a state of the sanitizing based on the analyzing of the sanitation data. Further, the processing unit 1010 of the sanitizing device 1000 may be configured for generating the at least one second input based on the determining of the state of the sanitizing. Further, the communication unit 1008 of the sanitizing device 1000 may be configured for transmitting the at least one first input to the communication unit 906 of the PCA system 900.

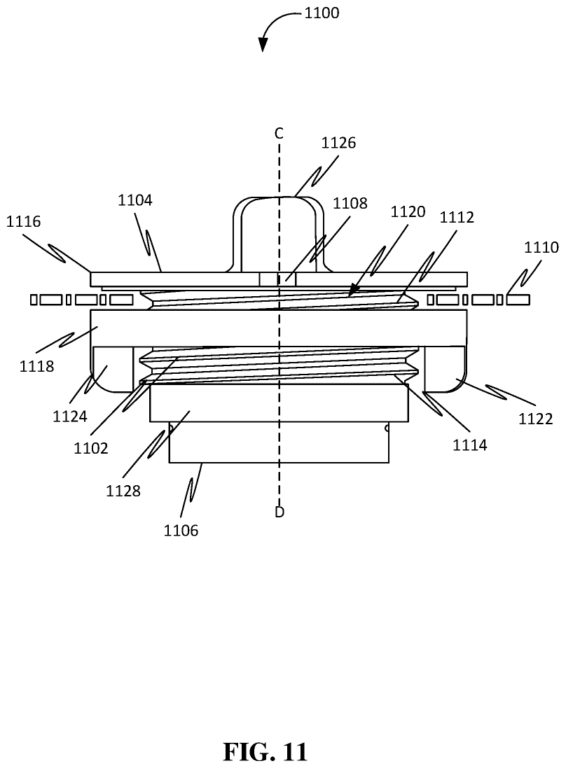

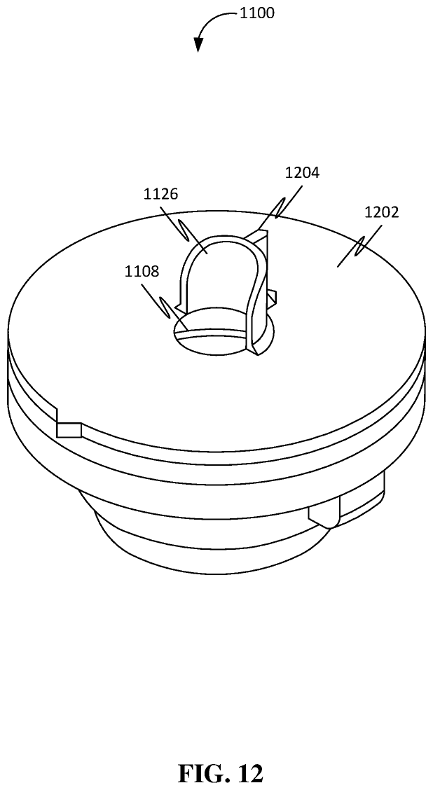

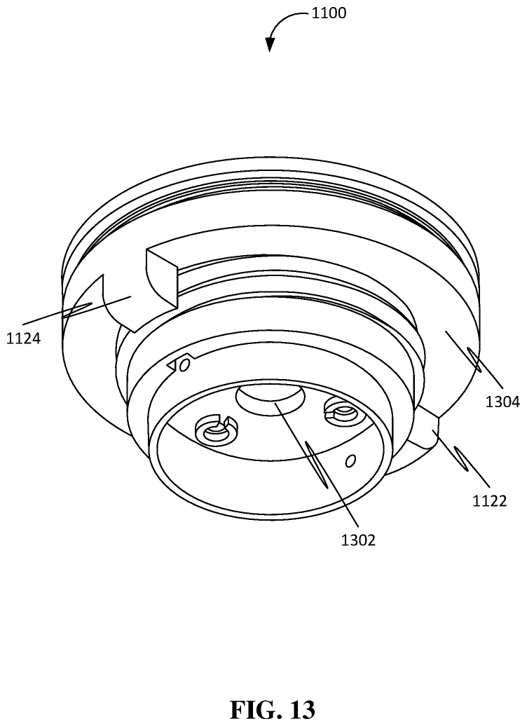

FIG. 11 is a front view of a device 1100 for facilitating sanitizing interior spaces of an aircraft using a pre-conditioned air (PCA) system associated with the aircraft, in accordance with some embodiments. Further, the device 1100 may be configured to be installed in a PCA supply duct 1110 around an opening in a duct wall of the PCA supply duct 1110. Further, the device 1100 may include a device body 1102 extending between a first end 1104 of the device 1100 and a second end 1106 of the device 1100. Further, the device body 1102 may include an interior cavity extending between the first end 1104 and the second end 1106. Further, the device 1100 may include an outlet opening 1108 disposed on a first end portion 1112 of the device body 1102 and an inlet opening 1302, as shown in FIG. 13, disposed on a second end portion 1114 of the device body 1102. Further, the interior cavity fluidly couples the inlet opening 1302 to the outlet opening 1108. Further, the first end portion 1112 may be placed inside the PCA supply duct 1110 and the second end portion 1114 may be placed outside the PCA supply duct 1110 after the installing of the device 1100 in the PCA supply duct 1110. Further, the device 1100 may include an interior flange 1116. Further, the interior flange 1116 may be disposed on the first end portion 1112 around the first end 1104. Further, the interior flange 1116 may be configured to be inserted in the PCA supply duct 1110 through the opening. Further, the device 1100 may include a threaded nut 1118. Further, the threaded nut 1118 may be threadedly coupled with the device body 1102 of the device 1100. Further, the device body 1102 may be threaded. Further, the threaded nut 1118 and the interior flange 1116 define a channel 1120 between the threaded nut 1118 and the interior flange 1116. Further, the channel 1120 receives at least one circumferential portion of the duct wall around the opening. Further, the threaded nut 1118 may be configured to be moved on the device body 1102 using at least one thumb tab 1122-1124 from at least one first position to at least one second position for securing the at least one circumferential portion of the duct wall between the interior flange 1116 and the threaded nut 1118 after the receiving. Further, the at least one thumb tab 1122-1124 may be disposed on an exterior surface 1304, as shown in FIG. 13, of the threaded nut 1118. Further, the device 1100 may include a flow shield 1126 disposed on an exterior surface 1202, as shown in FIG. 12, of the interior flange 1116. Further, the flow shield 1126 may be disposed on the first end portion 1112 around at least a part of a periphery of the outlet opening 1108. Further, the flow shield 1126 directs a flow of the at least one sanitizing agent in a direction for orienting the flow of the at least one sanitizing agent parallel to a flow of the PCA. Further, the flow shield 1126 may include a flow shield reinforcing support 1204 incorporated to a rear side of the flow shield 1126.

Further, the device 1100 may include a quick release coupling 1128. Further, the quick release coupling 1128 may be disposed on the second end portion 1114. Further, the quick release coupling 1128 may be configured to be engaged with a device outlet port of a sanitizing device.

FIG. 12 is a top perspective view of the device 1100, in accordance with some embodiments.

FIG. 13 is a bottom perspective view of the device 1100, in accordance with some embodiments.

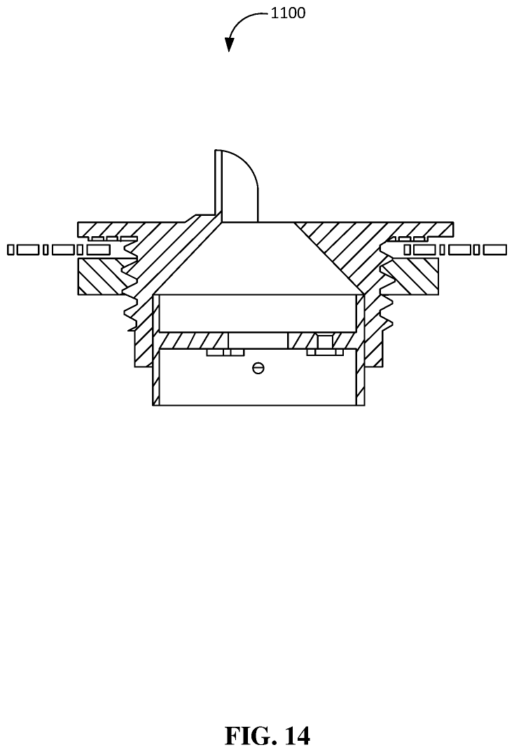

FIG. 14 is a right side cross-sectional view, along a section line C-D, of the device 1100 of FIG. 11, in accordance with some embodiments.

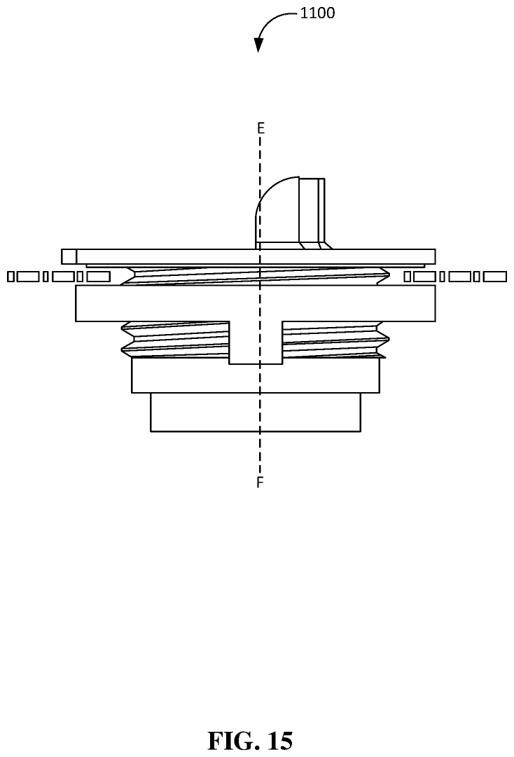

FIG. 15 is a left side cross-sectional view of the device 1100, in accordance with some embodiments.

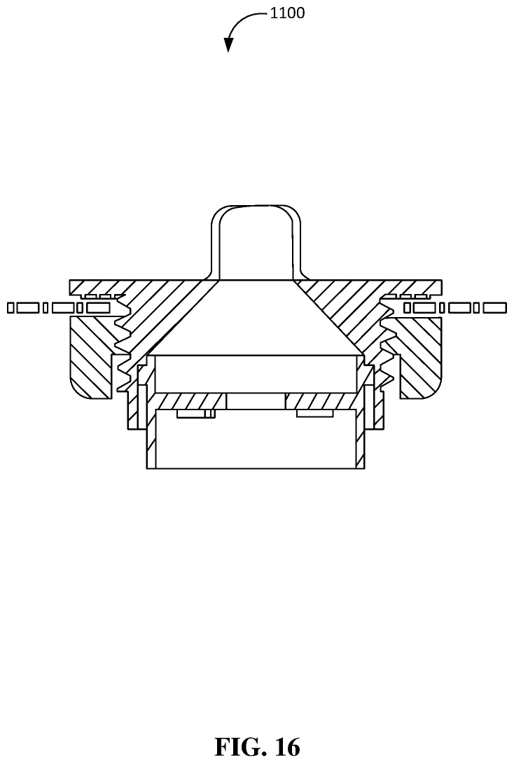

FIG. 16 is a front cross-sectional view, along a section line E-F, of the device 1100 of FIG. 15, in accordance with some embodiments.

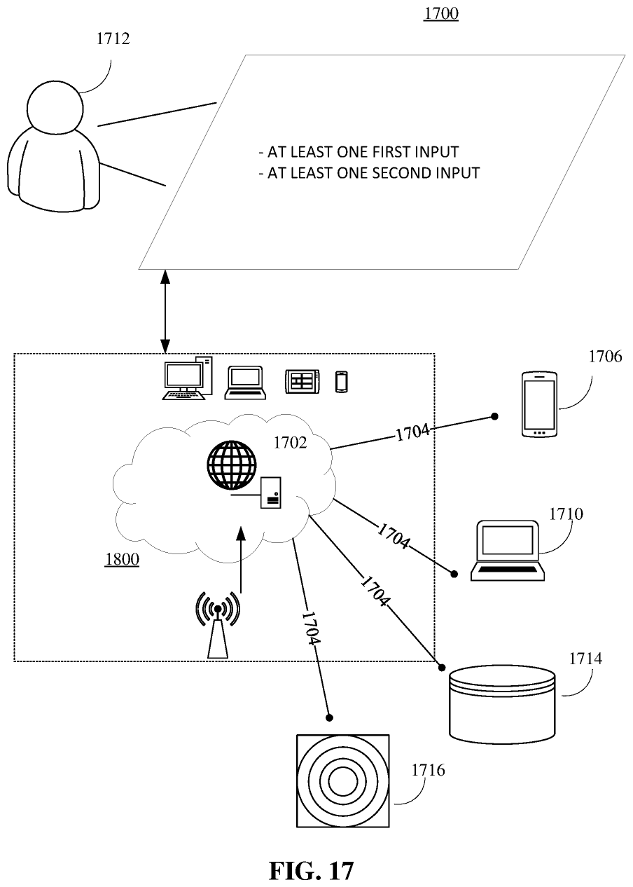

FIG. 17 is an illustration of an online platform 1700 consistent with various embodiments of the present disclosure. By way of non-limiting example, the online platform 1700 to facilitate sanitizing interior spaces of an aircraft using a pre-conditioned air (PCA) system may be hosted on a centralized server 1702, such as, for example, a cloud computing service. The centralized server 1702 may communicate with other network entities, such as, for example, a mobile device 1706 (such as a smartphone, a laptop, a tablet computer, etc.), other electronic devices 1710 (such as desktop computers, server computers, etc.), databases 1714, and sensors 1716 over a communication network 1704, such as, but not limited to, the Internet. Further, users of the online platform 1700 may include relevant parties such as, but not limited to, end-users, administrators, installers, and so on. Accordingly, in some instances, electronic devices operated by the one or more relevant parties may be in communication with the platform.

A user 1712, such as the one or more relevant parties, may access online platform 1700 through a web based software application or browser. The web based software application may be embodied as, for example, but not be limited to, a website, a web application, a desktop application, and a mobile application compatible with a computing device 1800.

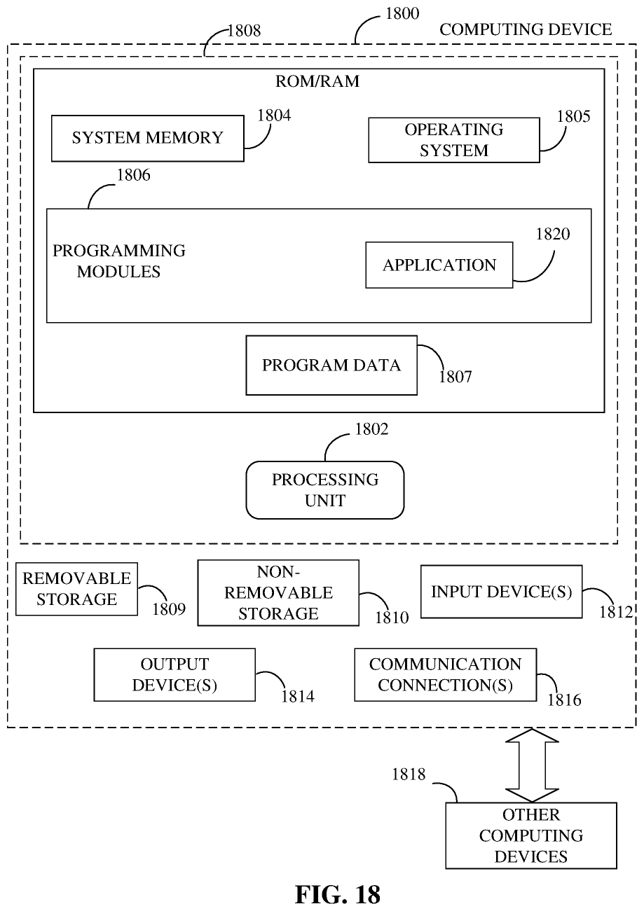

With reference to FIG. 18, a system consistent with an embodiment of the disclosure may include a computing device or cloud service, such as computing device 1800. In a basic configuration, computing device 1800 may include at least one processing unit 1802 and a system memory 1804. Depending on the configuration and type of computing device, system memory 1804 may comprise, but is not limited to, volatile (e.g. random-access memory (RAM)), non-volatile (e.g. read-only memory (ROM)), flash memory, or any combination. System memory 1804 may include operating system 1805, one or more programming modules 1806, and may include a program data 1807. Operating system 1805, for example, may be suitable for controlling computing device 1800's operation. In one embodiment, programming modules 1806 may include image-processing module, machine learning module. Furthermore, embodiments of the disclosure may be practiced in conjunction with a graphics library, other operating systems, or any other application program and is not limited to any particular application or system. This basic configuration is illustrated in FIG. 18 by those components within a dashed line 1808.

Computing device 1800 may have additional features or functionality. For example, computing device 1800 may also include additional data storage devices (removable and/or non-removable) such as, for example, magnetic disks, optical disks, or tape. Such additional storage is illustrated in FIG. 18 by a removable storage 1809 and a non-removable storage 1810. Computer storage media may include volatile and non-volatile, removable and non-removable media implemented in any method or technology for storage of information, such as computer-readable instructions, data structures, program modules, or other data. System memory 1804, removable storage 1809, and non-removable storage 1810 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 1800. Any such computer storage media may be part of device 1800. Computing device 1800 may also have input device(s) 1812 such as a keyboard, a mouse, a pen, a sound input device, a touch input device, a location sensor, a camera, a biometric sensor, etc. Output device(s) 1814 such as a display, speakers, a printer, etc. may also be included. The aforementioned devices are examples and others may be used.

Computing device 1800 may also contain a communication connection 1816 that may allow device 1800 to communicate with other computing devices 1818, such as over a network in a distributed computing environment, for example, an intranet or the Internet. Communication connection 1816 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 1804, including operating system 1805. While executing on processing unit 1802, programming modules 1806 (e.g., application 1820 such as a media player) may perform processes including, for example, one or more stages of methods, algorithms, systems, applications, servers, databases as described above. The aforementioned process is an example, and processing unit 1802 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the present disclosure has been explained in relation to its preferred embodiment, it is to be understood that many other possible modifications and variations can be made without departing from the spirit and scope of the disclosure.

What is claimed is:

1. A method for facilitating sanitizing interior spaces of an aircraft using a pre-conditioned air (PCA) system associated with the aircraft, the method comprising:
cutting an opening in a duct wall of a pre-conditioned air (PCA) supply duct of the PCA system, wherein the PCA supply duct supplies a pre-conditioned air (PCA) to at least one interior space of the aircraft from a pre-conditioned air (PCA) generator of the PCA system, wherein the aircraft is on the ground;
installing a device in the PCA supply duct around the opening, wherein the device comprises a device body extending between a first end of the device and a second end of the device, wherein the device body comprises an interior cavity extending between the first end and the second end, wherein the device comprises an outlet opening disposed on a first end portion of the device body and an inlet opening disposed on a second end portion of the device body, wherein the interior cavity fluidly couples the inlet opening to the outlet opening, wherein the first end portion is placed inside the PCA supply duct and the second end portion is placed outside the PCA supply duct based on the installing;
coupling the inlet opening of the device with a device outlet port of a sanitizing device after the installing, wherein a sanitizing agent generator of the sanitizing device is configured for generating at least one sanitizing agent for transferring the at least one sanitizing agent to the inlet opening of the device through the device outlet port; and
injecting the at least one sanitizing agent into the PCA supply duct from the outlet opening of the device based on the transferring, wherein the at least one sanitizing agent mixes with the PCA based on the injecting, wherein the at least one sanitizing agent is supplied to the at least one interior space along with the PCA for sanitizing the at least one interior space.

2. The method of claim 1, wherein the device comprises a cutting tool disposed on the first end portion, wherein the cutting tool is used for the cutting of the opening in the duct wall.

3. The method of claim 1 further comprising:
inserting an interior flange of the device in the PCA supply duct through the opening, wherein the interior flange is disposed on the first end portion around the first end;
receiving at least one circumferential portion of the duct wall around the opening into a channel between the interior flange and a threaded nut of the device after the inserting, wherein the threaded nut is threadedly coupled with the device body of the device, wherein the device body is threaded; and
moving the threaded nut on the device body using at least one thumb tab from at least one first position to at least one second position for securing the at least one circumferential portion of the duct wall between the interior flange and the threaded nut after the receiving, wherein the at least one thumb tab is disposed on an exterior surface of the threaded nut, wherein the installing of the device in the PCA supply duct is further based on the securing.

4. The method of claim 3 further comprising rotating the device between a plurality of positions after the inserting of the interior flange for orienting a flow shield of the device towards an inlet port of the aircraft, wherein the flow shield directs a flow of the at least one sanitizing agent in a direction, wherein the orienting of the flow shield orients the flow of the at least one sanitizing agent parallel to a flow of the PCA, wherein the flow shield is disposed on the first end portion around at least a part of a periphery of the outlet opening, wherein a duct outlet of the PCA supply duct is coupled to the inlet port of the aircraft, wherein the moving of the threaded nut on the device body is further based on the rotating.

5. The method of claim 1, wherein the coupling of the inlet opening with the device outlet port of the sanitizing device comprises engaging the device outlet port of the sanitizing device to a quick release coupling of the device, wherein the quick release coupling is disposed on the second end portion.

6. The method of claim 1 further comprising removing at least one waste from the at least one interior space of the aircraft, wherein the injecting of the at least one sanitizing agent into the PCA supply duct is further based on the removing of the at least one waste.

7. The method of claim 1 further comprising opening a door of each of at least one cabinet disposed in the at least one interior space of the aircraft, wherein the injecting of the at least one sanitizing agent into the PCA supply duct is further based on the opening of the door of each of the at least one cabinet.

8. The method of claim 1 further comprising transitioning at least one ventilation system of the aircraft to an open state, wherein the injecting of the at least one sanitizing agent into the PCA supply duct is further based on the transitioning of the ventilation system to the open state.

9. The method of claim 1 further comprising switching an air conditioning system of the aircraft to an off state, wherein the injecting of the at least one sanitizing agent into the PCA supply duct is further based on the switching of the air conditioning system to the off state.

10. The method of claim 1 further comprising:
receiving, using a communication unit of the sanitizing device, a contamination data associated with a contamination of the at least one interior space from at least one sensor disposed in the at least one interior space of the aircraft;
analyzing, using a processing unit of the sanitizing device, the contamination data;
determining, using the processing unit of the sanitizing device, at least one characteristic of the contamination of the at least one interior space based on the analyzing;
determining, using the processing unit of the sanitizing device, at least one parameter for the at least one sanitizing agent based on the at least one characteristic; and
generating, using the processing unit of the sanitizing device, at least one command for the sanitizing agent generator based on the determining of the at least one parameter, wherein the at least one command corresponds to the at least one parameter, wherein the generating of the at least one sanitizing agent is further based on the at least one command.

11. The method of claim 10, wherein the at least one parameter comprises a droplet size of a plurality of droplets of the at least one sanitizing agent, wherein the generating of the at least one command is further based on the droplet size of the plurality of droplets of the at least one sanitizing agent, wherein the generating of the at least one sanitizing agent comprises generating the plurality of droplets of the at least one sanitizing agent having the droplet size based on the at least one command.

12. The method of claim 10, wherein the at least one parameter comprises an application duration of the at least one sanitizing agent, wherein the generating of the at least one command is further based on the application duration of the at least one sanitizing agent, wherein the generating of the at least one sanitizing agent comprises generating the at least one sanitizing agent for the application duration based on the at least one command.

13. The method of claim 10, wherein the at least one parameter comprises a sanitizing agent identifier of a sanitizing agent of the at least one sanitizing agent, wherein the generating of the at least one command is further based on the sanitizing agent identifier, wherein the generating of the at least one sanitizing agent comprises generating the sanitizing agent based on the at least one command.

14. The method of claim 1 further comprising:
receiving, using a communication unit of the PCA system, at least one first input;
generating, using the pre-conditioned air (PCA) generator of the PCA system, the PCA based on the at least one first input, wherein the PCA supply duct supplies the PCA based on the generating of the PCA, wherein the PCA comprises at least one amount of moisture;
receiving, using the communication unit of the PCA system, at least one second input; and
generating, using the pre-conditioned air (PCA) generator, a first pre-conditioned air (PCA) based on the at least one second input, wherein the first PCA does not comprise the at least one amount of moisture, wherein the PCA supply duct supplies the first PCA to the at least one interior space of the aircraft based on the generating of the first PCA.

15. A method for facilitating sanitizing interior spaces of an aircraft using a pre-conditioned air (PCA) system associated with the aircraft, the method comprising:
cutting an opening in a duct wall of a pre-conditioned air (PCA) supply duct of the PCA system, wherein the PCA supply duct supplies a pre-conditioned air (PCA) to at least one interior space of the aircraft from a pre-conditioned air (PCA) generator of the PCA system, wherein the aircraft is on the ground;
installing a device in the PCA supply duct around the opening, wherein the device comprises a device body extending between a first end of the device and a second end of the device, wherein the device body comprises an interior cavity extending between the first end and the second end, wherein the device comprises an outlet opening disposed on a first end portion of the device body and an inlet opening disposed on a second end portion of the device body, wherein the interior cavity fluidly couples the inlet opening to the outlet opening, wherein the first end portion is placed inside the PCA supply duct and the second end portion is placed outside the PCA supply duct based on the installing;
coupling the inlet opening of the device with a device outlet port of a sanitizing device after the installing, wherein a sanitizing agent generator of the sanitizing device is configured for generating at least one sanitizing agent for transferring the at least one sanitizing agent to the inlet opening of the device through the device outlet port;
injecting the at least one sanitizing agent into the PCA supply duct from the outlet opening of the device based on the transferring, wherein the at least one sanitizing agent mixes with the PCA based on the injecting, wherein the at least one sanitizing agent is supplied to the at least one interior space along with the PCA for sanitizing the at least one interior space;
receiving, using a communication unit of the sanitizing device, a contamination data associated with a contamination of the at least one interior space from at least one sensor disposed in the at least one interior space of the aircraft;
analyzing, using a processing unit of the sanitizing device, the contamination data;
determining, using the processing unit, at least one characteristic of the contamination of the at least one interior space based on the analyzing;
determining, using the processing unit of the sanitizing device, at least one parameter for the at least one sanitizing agent based on the at least one characteristic; and
generating, using the processing unit of the sanitizing device, at least one command for the sanitizing agent generator based on the determining of the at least one parameter, wherein the at least one command corresponds to the at least one parameter, wherein the generating of the at least one sanitizing agent is further based on the at least one command.

16. The method of claim 15, wherein the coupling of the inlet opening with the device outlet port of the sanitizing device comprises engaging the device outlet port of the sanitizing device to a quick release coupling of the device, wherein the quick release coupling is disposed on the second end portion.

17. The method of claim 15 further comprising opening a door of each of at least one cabinet disposed in the at least one interior space of the aircraft, wherein the injecting of the at least one sanitizing agent into the PCA supply duct is further based on the opening of the door of each of the at least one cabinet.

18. The method of claim 15 further comprising transitioning at least one ventilation system of the aircraft to an open state, wherein the injecting of the at least one sanitizing agent into the PCA supply duct is further based on the transitioning of the ventilation system to the open state.

19. The method of claim 15 further comprising switching an air conditioning system of the aircraft to an off state, wherein the injecting of the at least one sanitizing agent into the PCA supply duct is further based on the switching of the air conditioning system to the off state.

20. A method for facilitating sanitizing interior spaces of an aircraft using a pre-conditioned air (PCA) system associated with the aircraft, the method comprising:
cutting an opening in a duct wall of a pre-conditioned air (PCA) supply duct of the PCA system, wherein the PCA supply duct supplies a pre-conditioned air (PCA) to at least one interior space of the aircraft from a pre-conditioned air (PCA) generator of the PCA system, wherein the aircraft is on the ground;
installing a device in the PCA supply duct around the opening, wherein the device comprises a device body extending between a first end of the device and a second end of the device, wherein the device body comprises an interior cavity extending between the first end and the second end, wherein the device comprises an outlet opening disposed on a first end portion of the device body and an inlet opening disposed on a second end portion of the device body, wherein the interior cavity fluidly couples the inlet opening to the outlet opening, wherein the first end portion is placed inside the PCA supply duct and the second end portion is placed outside the PCA supply duct based on the installing;
coupling the inlet opening of the device with a device outlet port of a sanitizing device after the installing, wherein a sanitizing agent generator of the sanitizing device is configured for generating at least one sanitizing agent for transferring the at least one sanitizing agent to the inlet opening of the device through the device outlet port;
injecting the at least one sanitizing agent into the PCA supply duct from the outlet opening of the device based on the transferring, wherein the at least one sanitizing agent mixes with the PCA based on the injecting, wherein the at least one sanitizing agent is supplied to the at least one interior space along with the PCA for sanitizing the at least one interior space;
receiving, using a communication unit of the PCA system, at least one first input;
generating, using the pre-conditioned air (PCA) generator of the PCA system, the PCA based on the at least one first input, wherein the PCA supply duct supplies the PCA based on the generating of the PCA, wherein the PCA comprises at least one amount of moisture;
receiving, using the communication unit of the PCA system, at least one second input; and
generating, using the pre-conditioned air (PCA) generator, a first pre-conditioned air (PCA) based on the at least one second input, wherein the first PCA does not comprise the at least one amount of moisture, wherein the PCA supply duct supplies the first PCA to the at least one interior space of the aircraft based on the generating of the first PCA.
